(19) United States
(12) Patent Application Publication
(10) Pub. No.: US 2009/0313038 A1
(43) Pub. Date: Dec. 17, 2009

(54) Method for Processing a Link of Time Segments

(75) Inventor: Geert Machtelinck, Mortsel (BE)

Correspondence Address: HOUSTON ELISEEVA, 4 MILITIA DRIVE, SUITE 4, LEXINGTON, MA, 02421, US

(73) Assignee: QUADRAT, Mortsel (BE)

(21) Appl. No.: 11/817,649

(22) PCT Filed: Feb. 17, 2006
(86) PCT No.: PCT/EP2006/060044

§ 371 Date: Sep. 20, 2007

Related U.S. Application Data
(60) Provisional application No. 60/666,001, filed on Mar. 29, 2005.

(30) Foreign Application Priority Data
Mar. 4, 2005 (EP) 05101703.6

(51) Int. Cl. G06Q 10/00; G06Q 50/00

(52) U.S. Cl. 705/2; 705/8

(57) ABSTRACT

A set of linked lists of time segments represent the actions of resources that are input of a time scheduling system. A system and method are described that operate on said linked list and that produce a solution that meets the constraints imposed by the actions of said resources. The method is described in the context of appointment scheduling systems for medical institutions, where appointments need to be scheduled for patients, taking into account a multitude of constraints such as the availability of staff, equipment, facilities and of the patient himself .

### FIELD OF THE INVENTION

The present invention relates to a system and a method for the scheduling of resources, more specifically for automated scheduling of patients, personnel, equipment and facilities in medical institutions taking into account operational constraints.

### BACKGROUND OF THE INVENTION

Traditional project management tools describe large scale projects as tasks and subtasks. Certain tasks have to be scheduled sequentially, while other tasks can be performed concurrently. With each task a duration time is associated which can be fixed or variable. The task of a project management tool is to identify the optimal sequence of tasks and subtasks in a way that the duration for executing the complete project is minimized.

A well known scheduling technique that is described in the literature on management techniques is called PERT (Program Evaluation and Review Technique), which provides a formalism to represent the tasks and subtasks in a project by means of a network diagram. PERT provides also a method to determine the critical path, which is that sequence of tasks that directly impact the duration of the project as a whole. Variations on PERT are described that model the effect on the critical path of deviations from the original schedules.

Gantt developed a visual tool to represent the progress of activities in a diagram using vertical bars and links. Such a diagram is called a GANTT chart.

PERT and GANTT charts provide powerful techniques to manage large scale projects. Their strength is that they enable to distinguish tasks of which the duration directly impacts the duration of the project from other tasks where some slack time is permitted.

In the context of scheduling appointments for patients and the use of other resources such as diagnostic or treatment equipment and examination rooms in a hospital environment, optimizing the critical path does not always yield the most desirable solution. For example, assuming that there is no medical urgency involved, a patient may prefer having an exam performed at a later time by a medical doctor whom he knows, rather than having the same exam done at an earlier time by a medical doctor whom he doesn't know. Other preferences may include a morning versus an afternoon appointment, an appointment at a particular facility etc. The medical doctor may also have his preferences. For example he may prefer certain equipment, or an examination or treatment room he is familiar with.

To enable a patient or a medical staff member to make a preferred choice from a set of possible appointments, a system is needed that first calculates what the different alternatives are. In other words, a scheduling system is needed that does not come up with just one proposal to book the use of a set of resources and that is the soonest one available, but that calculates a set of alternative solutions.

PERT, GANTT charts and their variations are implicitly designed to optimize the critical path in a project, including taking into account variations of the duration individual tasks. When used as a tool for the purpose of scheduling the use of resources, they will produce one single solution that corresponds with the soonest available booking of that resource. If more than one solution is needed, the method can be run again with different constraints. This approach is time consuming and inefficient from the viewpoint of number of calculations that are required. Furthermore the PERT and GANTT techniques are actually designed to plan activities, rather than for managing the complexity of scheduling tasks and subtasks that are constraint by the availability of resources.

A possible solution to the above problem would be to initialize a time counter at a certain value and to check if a given resource can be scheduled at the time of the counter taking into account the constraints imposed by the availability of the other resources. By incrementing the time counter in small steps and checking every time again if the resource can be booked, it is possible to obtain an overview of the time segments that are available for booking said resource. This method works fine, but requires many calculations, since for every time increment the constraints imposed by the availability of the resources need to be consulted and processed. This results in a slow response of the scheduling system. Furthermore, the use of a finite time increment may result in inaccuracy of the exact start and end times of the availability of resources, due to rounding off errors.

### SUMMARY OF THE INVENTION

From the above follows that a new scheduling method is needed that is capable to calculate not just one solution but a set of multiple alternative solutions for the management of resources that are dependent on the availability of other resources.

The above-mentioned problems are solved by a system and a method as set forth in claims 1 and the other dependent and independent claims.

### BRIEF DESCRIPTION OF THE DRAWING

FIG. 1 describes a set of actions related to resources and connected by comprising, relational and sequential links;

FIG. 2 describes a reduced set of actions that is left after working out the relational links according to a preferred embodiment;

FIG. 3 describes a reduced set of actions that is left after working out the relational and comprising links according to a preferred embodiment;

FIG. 4 describes a reduced set of actions that is left over after working out the relational, comprising and sequential links according to a preferred embodiment;

FIG. 5 describes a set of time windows associated with actions;

FIG. 6 demonstrates the processing of a relational link according to a preferred embodiment;

FIG. 7 demonstrates the processing of a comprising link according to a preferred embodiment;

FIG. 8 demonstrates the processing of a sequential link with a preceding action according to a preferred embodiment;

FIG. 9 demonstrates the processing of a sequential link with a following action according to a preferred embodiment;

FIG. 10 demonstrates the processing of a sequential link with a following action, taking into account slack time according to a preferred embodiment;

FIG. 11 shows an example of processing a relational link according to a preferred embodiment;

FIG. 12 shows another example of processing a relational link according to a preferred embodiment;

FIG. 13 shows three examples of processing a comprising link according to a preferred embodiment;

FIG. 14 shows an example of the processing of time windows according to a preferred embodiment;

FIG. 15 shows an example of using deductive logic;

FIG. 16 shows an example of using inductive logic;

FIG. 17 shows a data processing system according to a preferred embodiment of the current invention.

### DETAILED DESCRIPTION OF THE INVENTION

Before explaining the general principles of the method according to the current invention, the method is first explained by working out a specific example, which is also one specific embodiment of the current invention.

According to the example, an appointment needs to be scheduled to examine a patient by means of a scanner. The patient needs to undress before and to dress again after the scan.

The exam itself takes 2 hours. Both for undressing and dressing one hour is provided. After the patient has undressed, he does not want to wait for the exam. When the exam is finished, he accepts that he may have to wait up to one hour before he can dress again.

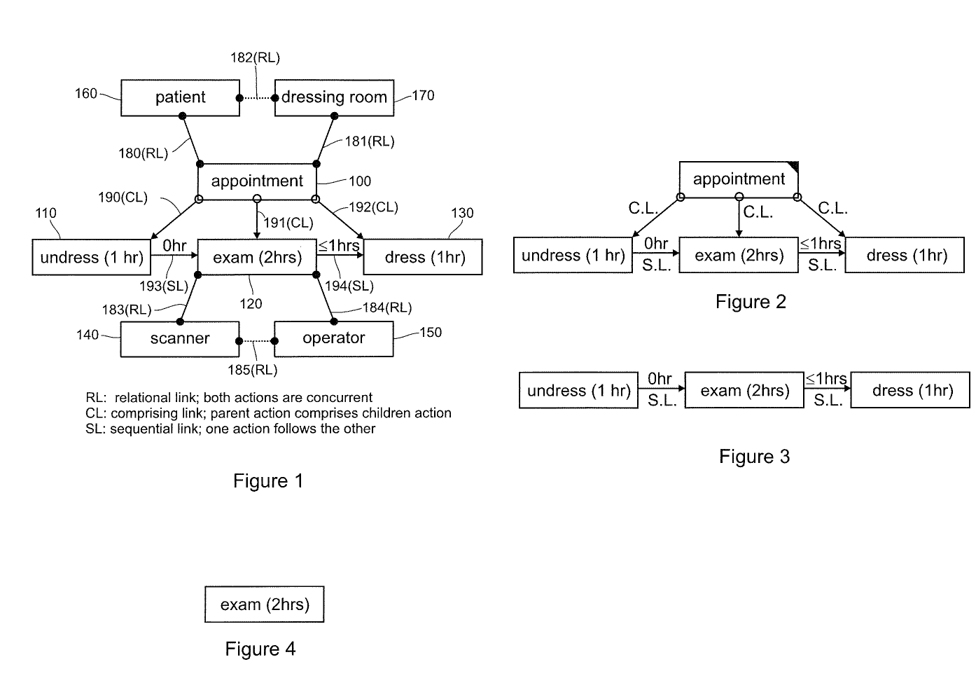

FIG. 1 describes the actions that are part of the appointment and the relations between them. The appointment (100) action comprises three other actions: the undressing (110) action, the actual exam (120) action and the dressing (130) action. This comprising relationship is represented by three comprising links (190, 191, 192) between the individual actions (110, 120, 130) and the appointment (100) action. The appointment (100) action is called a parent relative to the undressing (110), the actual exam (120) and dressing (130) actions which are called children. Because of the parent-child relationship of a comprising link (190, 191, 192), it is not symmetrical.

An action is defined as being “atomic” when it does not comprise other actions. For example, the undress (110) action is atomic, but the appointment (100) action is not.

The undressing (110), the actual exam (120) and dressing (130) actions follow sequentially and this relationship is represented by the sequential links (193, 194). The sequential nature implies that such a link is not symmetrical, as the arrows in FIG. 1 also indicate.

The exam (120) can only be carried out when the scanner (140) is available. This kind of relationship is represented by a relational link (183). In addition does carrying out the exam require the availability of an operator, so a relational link (184) also exists between the exam and the operator (150). A relational link between two actions indicates that both actions can only be carried out at the same time. From this follows that such a link is by nature symmetrical and transitive. The transitivity is expressed in FIG. 1 by the dotted line (185) between the scanner and operator action.

In a more general case, a procedure or exam is preceded by a pre-op action and followed by a post-op action. In a more general case an action refers to an activity related to a resource. Such a resource can be a patient, a physician, a nurse, an operator a diagnostic or treatment apparatus, a examination or treatment room, or any other kind of resource with which an activity can be associated. The resource can or can not be related to the domain of healthcare. The activity can be the use of equipment, the presence of a person, the occupation of a facility or any other activity that refers to the use or availability of any resource. In a more general case any topology of any number of actions related by comprising, relational or sequential links is possible.

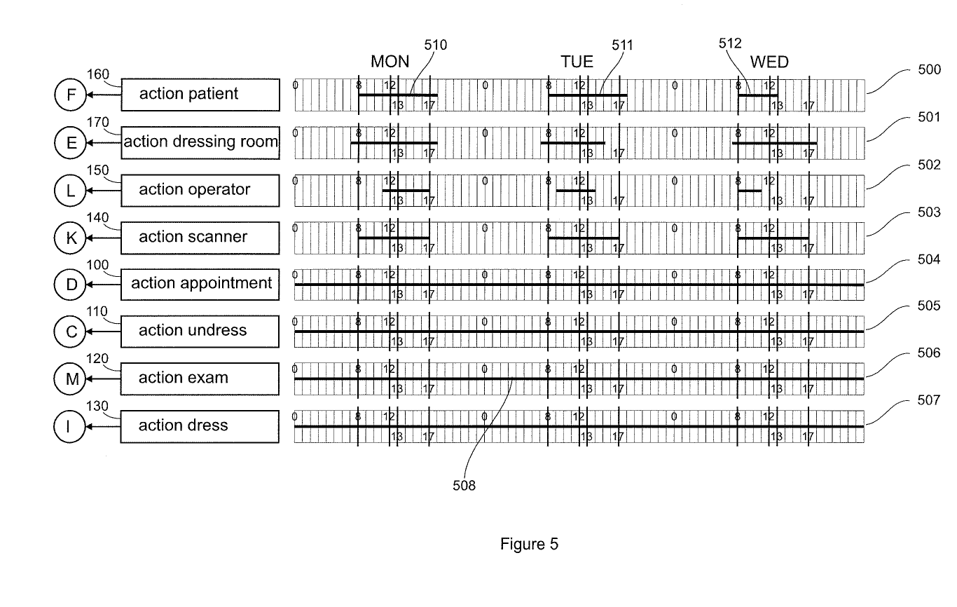

FIG. 5 shows how with each action (100, 110, 120, 130, 140, 150, 160, 170) in FIG. 1 a corresponding time window (501-507) is associated. A time window consists of a linked list of non contiguous time segments, each segment having a beginning and an ending time. For example, for the patient (160) action, the linked list consists of the time segments (510, 511, 512).

A time window can represent the range of time when an action can potentially occur. However, a time window can also represent a range of time when the action can start or when it can end.

In the example in FIG. 5, the time windows (500-503) of the patient (150), the dressing room (170), the scanner (140) and the operator (150) are part of the problem definition data. These time windows represent constraints imposed by the corresponding resources. The time windows (504-507) of the undressing (110), exam (120) and dressing (130) actions and of the appointment (100) as a whole, however, are initially undetermined, as they are the subject of the solution that has to be calculated for the scheduling problem. An undetermined time window is represented as one contiguous time segment with the length of the time window. For example, 508 is the initial time window associated with the exam action (120). As a solution for the time scheduling problem is being processed according to the current invention, the number of segments of an undetermined time window may change and the beginning and end times of the remaining time segments may become increasingly more focused, until they represent a situation that is consistent with all the constraints imposed by the resources.

Since the constraints imposed by the resources are represented by relational (180-185), comprising (190-192) and sequential (193, 194) links, processing the solution essentially comes down to working out these links.

When working out the links, a number of different cases are to be distinguished that correspond with the different nature of the links (relational, comprising or sequential), the interpretation of the time window of the action (start times, end times or action times), and the relative location of the time segments (the way that the time segments in the time windows of the linked actions overlap). The result of processing a link involves adjusting the time segments in the time windows corresponding to the linked actions in a way that they become consistent with the constraints imposed by the corresponding resources.

In the following paragraphs the processing of the different links is discussed.

First case: Time Window Processing for Actions Connected Through Relational Links

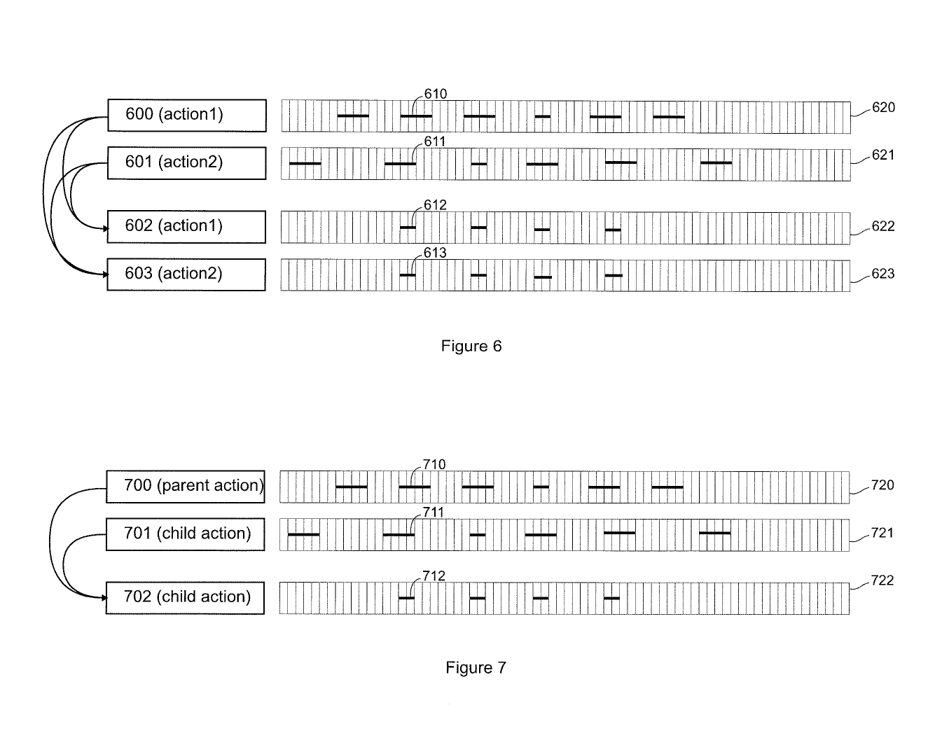

FIG. 6 illustrates a number of situations for actions connected through relational links, of which the time segments occur in different relative positions (overlapping and non-overlapping). The interpretation of the time windows (620-623) is that the represent the time during which the action (600-603) can take place. Since the meaning of a relational link is that the two actions (600,601) can only take place simultaneously, the effect of working out the link is that each time window (620,621) should be replaced by a time window (622,623) that consists of time segments (612,613) that are the cross sections of the time segments (610,611) in the original time windows.

Because of the transitive nature of a relational link, if an action has more than one relational link—directly or indirectly—to another action, the time windows of all the actions are to be replaced by a time window of which the time segments are the cross sections of all the time segments of the time windows of all the related actions.

Second case: Time Window Processing for Actions Connected Through Comprising Links

FIG. 7 illustrates a number of situations for actions connected through comprising links, of which the time segments occur in different relative positions (overlapping and non-overlapping). The interpretation of the time windows (700-702) is that the represent the time during which the action can take place. The meaning of a comprising link is that the time segments (711) of a child action (701) have to occur within the time segments (710) of the time window (720) of the parent action (700). This is achieved by replacing the time segments (711) of the time window (721) of the child action (701) by the cross section (712) of themselves (711) with the time segments (710) of the time window (720) of the parent action (700).

Third case: Time Window Processing for Actions Connected Through Sequential Links

The following terms are introduced or clarified:
time window of an action: linked list of time segments describing when an action can take place.
time window of start times of an action: linked list of time segments describing when said action can start;
time window of end times of an action: linked list of time segments describing when said action can end;

The time window of an action, the time window of start times of the same action and the time window of end times of that same action are interrelated.

Referring to FIG. 9 and according to an embodiment of the current invention, a time window (921) representing start times (911) of an action is calculated from a corresponding time window (920) representing said action, by subtracting from the end times of the time segments (910) in the latter time window (920) the duration (930) of said action.

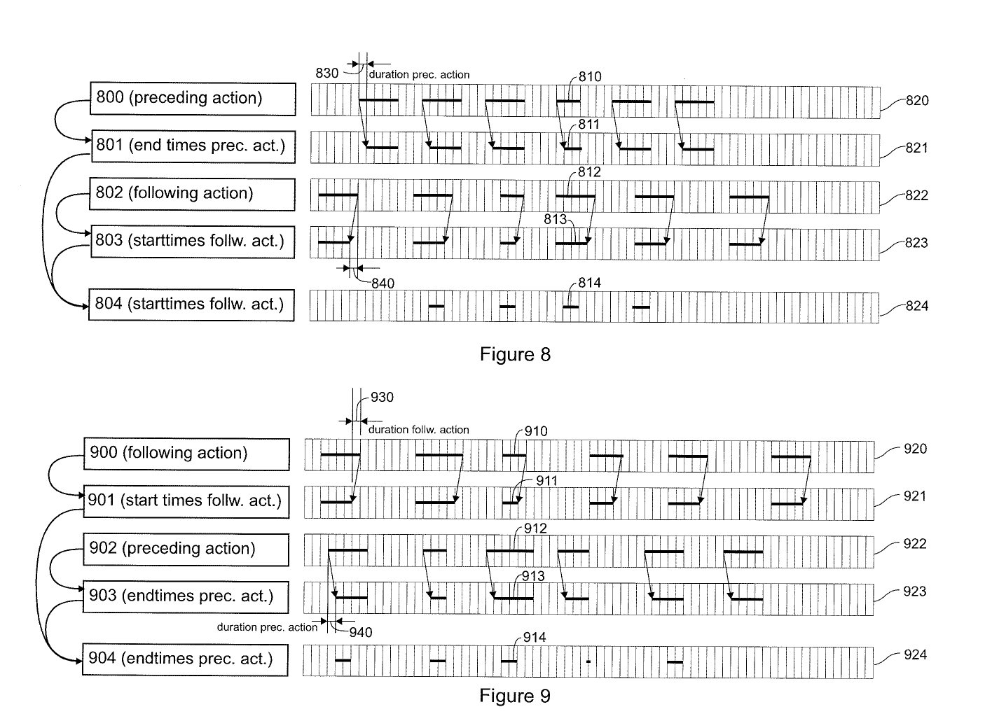

Referring to FIG. 8 and according to an embodiment of the current invention, a time window (821) representing end times is of an action is calculated from a corresponding time window (820) representing said action, by adding to the start times of the time segments (810) in the latter time window (820) the duration (830) of the action.

According to an embodiment of the current invention time windows representing start times and end times of an action are also interrelated by shifting the start and end times in the time segments by the duration of the action.

According to one embodiment of the current invention, when a first preceding action (800, 902) is followed by a second following action (802, 900), certain restrictions are applied on both the start and end times of both actions.

A first restriction involves the start times of a following action in order to achieve that that the start times of a following action can never be earlier than the earliest end time of any of the preceding actions. According to one aspect of the current invention, this effect is achieved by replacing the time segments (813) of the start times (823) of the following action (802) by the cross section (814) between themselves (813) and the time segments (811) of the end times (821) of the preceding action (800).

A second restriction involves the end times of the preceding action in order to achieve that the end times of a preceding action can never be later than the latest start times of any of the following actions. According to one aspect of the current invention, this effect is achieved by replacing the time segments (913) of the end times (923) of the preceding action (902) by a cross section (914) between themselves (913) and the time segments (911) of the start times (921) of the following action (900).

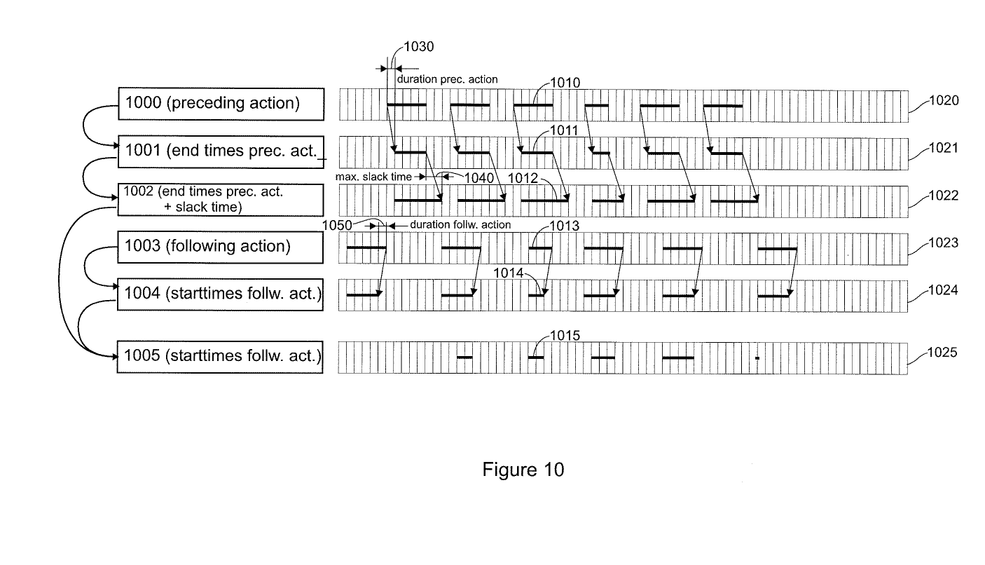

In the case that slack time is allowed between two actions, the end times of the time segments of the preceding action are preferably extended by the maximum allowed slack time, prior to applying said first restriction. Referring to FIG. 10, the time window (1020) of the preceding action (1000) is used to calculate the time window (1021) of the end times (1001) of the preceding action (1000) by shifting the start times of the time segments (1010) forward by the duration (1030) of the preceding action (1000). Following that, the segments (1011) of the time window (1021) of the end times (1001) of the preceding action are extended by the maximum slack time (1040) to yield the time segments (1012) of the time window (1022) of the end times (1002) of the preceding action plus the slack time. To obtain the time window (1024) of the start times of the following action (1004), the end times of the segments (1013) of the time window (1023) of the following action (1003) are shifted backwards by the duration (1050) of the following action (1003). The segments (1015) of the time window (1025) of the start times of the following action (1005) are obtained by making the cross section between the time segments (1012) and the time segments (1014).

Working out a sequential link between two actions involves applying the two above restrictions.

Having described how according to the current invention:
relational links are processed(1);
composite links are processed (2);
the relation between time windows representing actions, start times and end times (3) is processed;
sequential links are processed (4);
slack time is processed in sequential links (5).
   we proceed next by working out the example that was earlier introduced according to the principles of the current invention.

The problem that has to be resolved is finding the time window representing the start time(s) for the exam.

A first step consists of working out the relational links in FIG. 1.

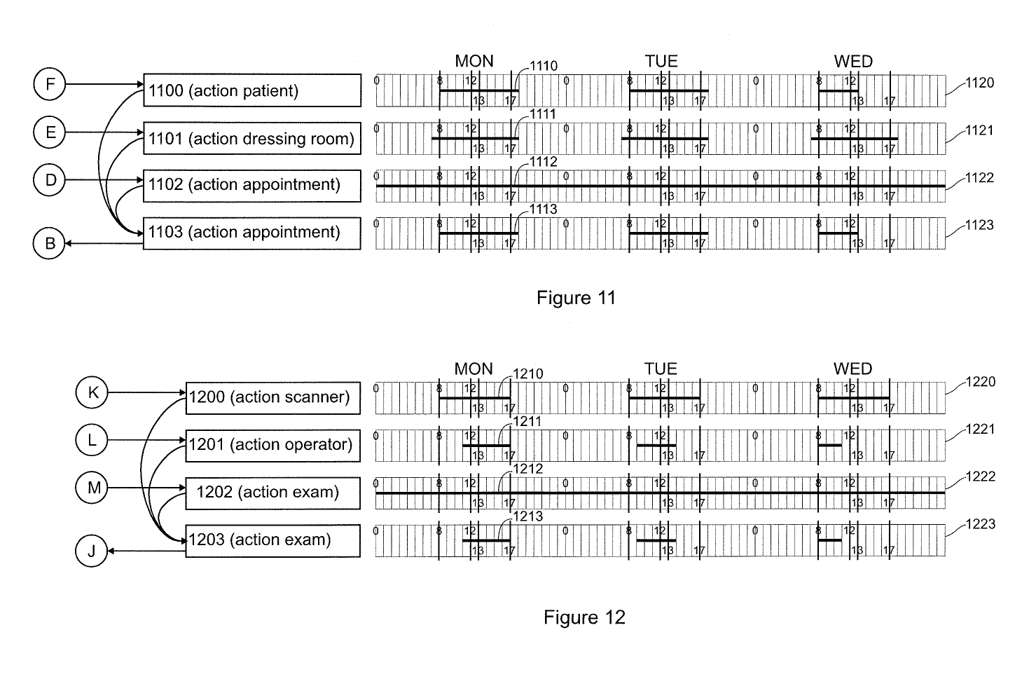

Referring to FIG. 11, this is done by using the general principles according to the current invention that were earlier explained by means of FIG. 6.

Similarly, referring to FIG. 12, the relational links can be worked out between the exam, the operator and the scanner.

After this operation, the graph in FIG. 1 can be reduced to the one in FIG. 2, with the notion that he time windows associated with the appointment and the exam actions are not the original ones, but the ones that were obtained from the previous step.

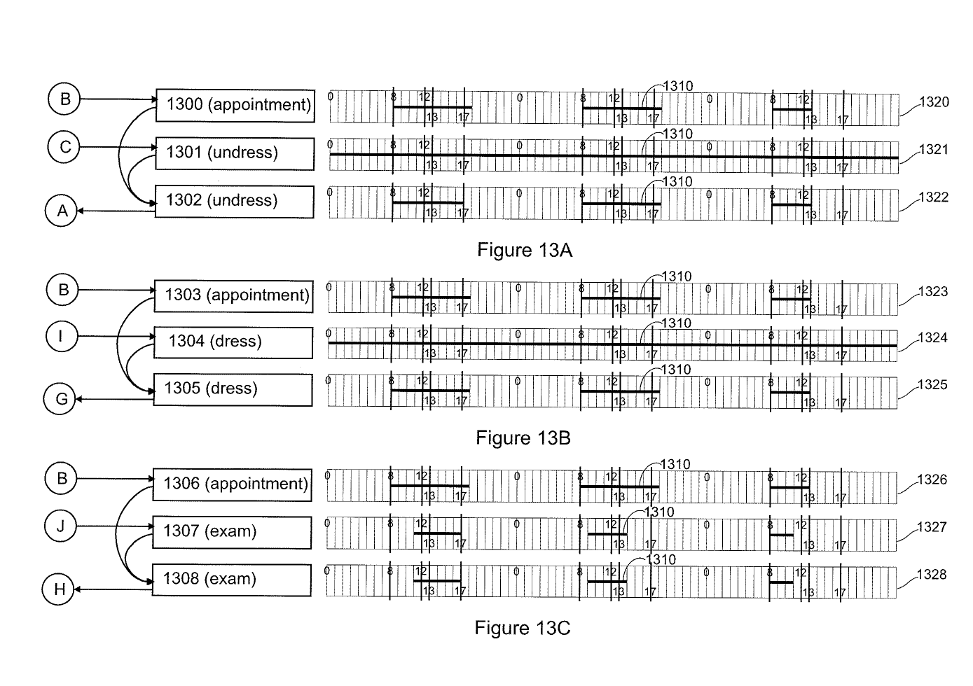

A second step consists of working out the comprising links in the graph in FIG. 2. According to the current invention, This is achieved by processing the time segments in the time windows of the undress, exam and dress actions so that they fall within the time segments of the time window of the appointment action. This is demonstrated in FIGS. 13A, 13B and 13C using the general principles of the current invention that were earlier explained by means of FIG. 7.

After this operation, the graph in FIG. 1 or FIG. 2 can be reduced to the one in FIG. 3, with the notion that the time windows associated with the undress, exam and dress actions are not the original ones, but the ones that were obtained from the previous step.

The third step consists of working out the constraints imposed by the sequential links.

The exam action is preceded and followed by another action. According to one aspect of the current invention, this has implications on start and end times of the time segments of the corresponding time windows.

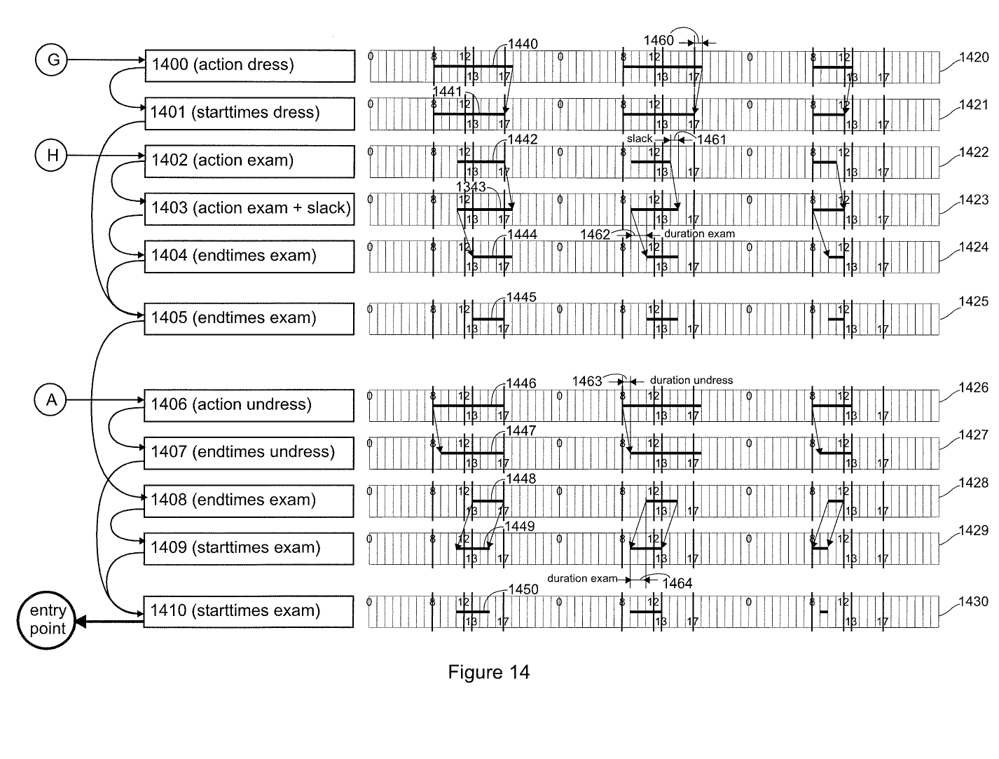

Referring to FIG. 14, the start times (1310) of the exam should never be earlier than the earliest end times (1307) of the undress action, and the end times (1303) of the exam including slack time should never be later than the latest start times (1301) of the dressing action, according to the general principles that were earlier explained by means of FIGS. 8, 9 and 10.

After this operation, the graph in FIGS. 1, 2 and 3 can be reduced to the one in FIG. 4, with the notion that the time window associated with the exam actions are the ones that were obtained from the previous step.

### Introducing Deductive and Inductive Logic

According to a preferred embodiment of the current invention, an inductive logic method is used to control the processing of the time windows as opposed to deductive logic. These terms are explained in more detail.

Generally speaking, deductive logic starts with variables of which the values are known (called “the hypotheses”) and deduces step by step according to a predefined flow the value of the variable for which a solution is sought (called the “final conclusion”). This processing occurs through the calculation of the value of intermediate values (called “intermediate conclusions”).

In deductive logic, the information processing flow itself is the subject of the programming and as a result, once it has been programmed, it is fixed. Therefore, deductive logic programming is efficient for those problems of which the taxonomy of relations between variables is fixed, and only the values of the hypotheses are subject to change.

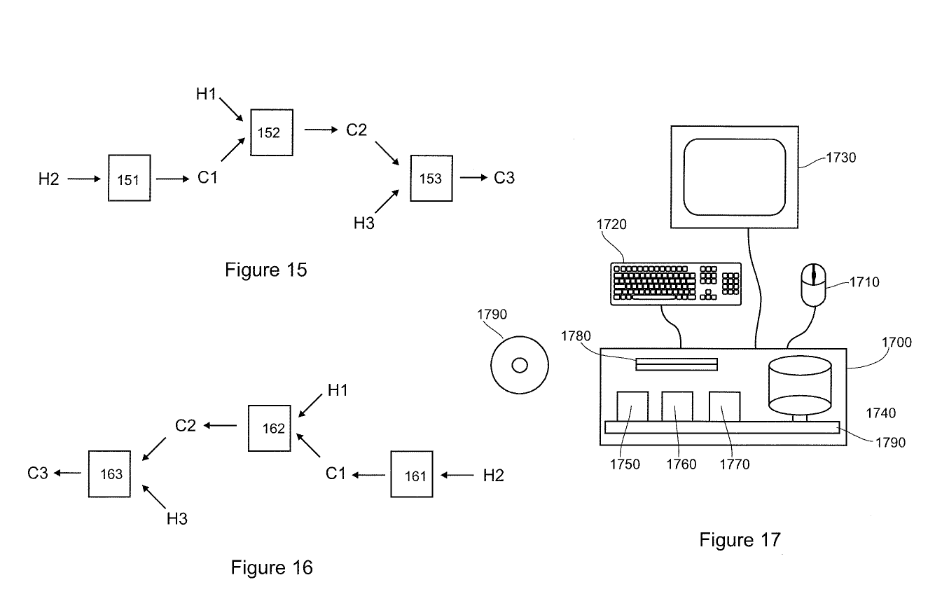

An example of a deductive logic method is shown in FIG. 15. H1, H2 and H3 are the basic hypotheses. Processing (151) the hypothesis H2 results in the intermediate conclusion C1. Processing (152) the conclusion C1 and the hypothesis H1 results in the intermediate conclusion C2. Processing (153) the conclusion C2 and the hypothesis H3 then leads to the final conclusion C3.

In contrary, the entry point for an inductive logic method according to the current invention is the final conclusion itself of which the value is initially unknown. By means of a set of inductive steps that take the form of an exploration process, the data of the hypotheses is first gathered and then systematically processed to calculate the final conclusion.

An inductive step to calculate an (intermediate) conclusion comprises determining what other variables are needed to calculate said (intermediate) conclusion. There are two possibilities:
1) Either the values of the variables that are needed are known because they are either hypotheses or intermediate conclusions of which the value has been earlier determined; in that case the variables can be processed to obtain the (intermediate) conclusion.
2) Or at least one of the variables that are needed is an intermediate conclusion of which the value has not been determined yet; in that case this (intermediate) conclusion initiates a new inductive step.

The subject of the programming in an inductive logic method is not a deductive information processing flow, but a rule set that manages the inductive steps.

Developing a rule set for an inductive method involves determining:
1) the nature (classes) of the variables (intermediate conclusions) that are needed to calculate a conclusion;
2) for each nature (class) of a variable (intermediate conclusion) determining what kind of processing on what other variables (other intermediate conclusions or hypotheses) is needed to calculate the result of said (intermediate) conclusion.

Unlike in a deductive logic method, the problem definition now not only states the values of the hypothesis, but also the taxonomy of the relations between the variables. This allows for far greater flexibility when solving problems that have different taxonomies of relations between variables. Once the rule set has been programmed, problems with a wide variety of taxonomies of relations between the above variables can be solved using the same program.

An example of using an inductive logic method is presented in FIG. 16. The entry point is a call to calculate the value of the variable C3. The rule set dictates that the variable C3 requires the processing of two other variables being H3, of which the value is known since it is a hypothesis, and the intermediate conclusion C2, of which the value at this point is unknown. The latter causes a new inductive step to calculate the unknown variable C2. The rule set dictates that the variable C2 requires the processing of two other variables H1, of which the value is known since it is a hypothesis, and of the intermediate conclusion C1, of which the value at this point is unknown. The latter causes a new inductive step to calculate C1. The rule set dictates that the variable C1 requires the processing of the variable H2, of which the value is known. This results in the processing of H2 to obtain C1. Now that C1 is known, this results in the processing of C1 and H1 to calculate C2. Now that C2 is known, this results in the processing of C2 and H3 to calculate the final conclusion C3.

### Preferred Embodiment Based on Inductive Logic

According to the current invention, the solution of the scheduling problem stated in the above example is preferably carried out by using an inductive logic method.

According to one embodiment, the following classes or variables are used for managing resources:
time window related to an action
time window related to the start times of an action
time window related to the end times of an action

According to the same embodiment the inductive logic is managed by a set of three rules:
a first rule dictates that obtaining the value of a variable of the type “start times of an action” requires the processing of the value of the “end times of that action” and the value of “the previous action”.
a second rule dictates that obtaining the value of a variable of the type “action” requires the processing of the values of the “parent actions” and the “related actions”.
a third rule dictates that obtaining the value of a variable of the type “end times of an action” requires the processing of that same “action”, the “slack time” and “the following action”.

In a more general case other sets of rules can be selected that however yield equivalent results and also fall within the scope of the current invention. This follows from the fact that the classes of variables in the above rule set are related to each other by simple relationships.

We have found that the above set of three classes of variables in combination with the above three rules provides a self contained method than enables resource scheduling and management of a wide variety of situations.

The method according to the current invention processes time windows and results in a time window that generally comprises a plurality of time segments, each one indicating a single solution of when the corresponding action can take place (or start). The method hence produces not just one solution for the scheduling problem, as in the prior art, but a complete set of solutions.

The method according to the current invention can be used for any resource scheduling and management problem that can be modeled as a set of actions corresponding to resources that are related by a combination of comprising, relating and sequential links and slack time.

Having described the general principles of the current invention we proceed by working out the example that was earlier introduced.

Referring to FIG. 14, the method starts by instantiating a variable start times exam, which is the final conclusion of the scheduling problem.

The symbols in the circles on one of the FIGS. 11 to 14 indicate references to the same symbols in circles in one of the other figures.

Since the value of the variable start times exam at this point is unknown, this induces an inductive step (IS1). The first rule according to the current invention dictates that in order to calculate the value (1410) of the start times of the exam, the values (1408=1405) of the end time of the exam action and (1406=1302) of the undress action are needed. Since none of these values are known at this time, this causes two new inductive steps: a first one (IS2) to enable the calculation of the value (1406=1302) of the undress action and a second one (IS3) for the calculation of the value (1408=1405) of the end times of the exam.

We proceed by first explaining the inductive step (IS2). Referring to FIG. 11 to 14, the second rule dictates that in order to calculate the value (1406=1302) of the undress action requires the processing of the value (1300=1103) of the appointment action which is the parent action. Since the value (1300=1103) of the appointment action is not known at this time, this induces again an inductive step (IS4) for the calculation of that variable. Since this variable (1300=1103) appointment is of the type “action”, the same (second) rule applies, requiring the processing of the values of related dressing room (1101) and patient (1100) actions. The values of these actions are known since they are hypotheses, so this enables to calculate the value of the appointment (1300=1103) action and subsequently of the undress (1406=1302) action.

We next proceed by describing the inductive step (IS3). Referring to FIG. 11 to 14, the third rule dictates that the calculation of the value (1408=1405) of the end times of the exam requires the processing of the value (1402=1308) of the exam action and of the value (1400=1305) of the dress action. Since the variable (1402=1308) of the exam action is of the type “action”, the second rule applies, and this requires the processing of the values of the parent appointment (1306=1103) action, and of the related scanner (1200) and operator (1201) actions. The value (1306=1103) of the parent appointment action is calculated the same way as in the inductive step (IS2). The values (1200, 1201) of the relating actions are known, since they are hypotheses, so this enables to calculate the value of the exam (1402=1308) action. Since the variable (1400=1305) is also of the type action, the second rule is applied once more, leading to the processing of the values of the variables (1303=1103) and (1304=1101). At this point the calculation of the value (1408=1405) of the end times of the exam can be completed and subsequently the calculation of the value (1410) of the start times of the exam.

The above mentioned invention is preferably implemented using a data processing system such as a computer. An embodiment of such a system (1700) is shown in FIG. 17. A computer comprises a network connection means (1750, a central processing unit (1760) and memory means (1770) which are all connected through a computer bus (1790). The computer typically also has a computer human interface for inputting data (1710, 1720) and a computer human interface for outputting data (1730). According to one embodiment, the computer program code is stored on a computer readable medium such as a mass storage device (1740) or a portable data carrier (1790) which is read by means of a portable data carrier reading means (1780).

Having described in detail preferred embodiments of the current invention, it will now be apparent to those skilled in the art that numerous modifications can be made therein without departing from the scope of the invention as defined in the appending claims.

1. A computerized method for scheduling actions corresponding to resources comprising the steps of:
defining relationships between the actions of said resources, said relationships belonging to the set of comprising, relational or sequential relationships;
identifying a first set of resources of which action constraints are known and a second set of resources of which action constraints are unknown;
describing the actions of the resources of said first and said second set of resources by means of time windows comprising a linked list of time segments;
identifying a resource out of said second set of resources;
processing said time windows to determine the action of said identified resource; wherein said processing comprises an inductive logic step.

2. The method according to claim 1, wherein said time windows belong to the set of an action, a start time of an action, or an end time of an action.

3. The method according to claim 1, wherein the inductive logic step comprises obtaining a time window of start times of an action by processing a time window of end times of said action and a time window of a previous action.

4. The method according to claim 3, wherein the inductive step of obtaining a time window of start times of an action by processing a time window of end times of said action and a time window of a previous action comprises the following steps:
adding the duration of said previous action to the start times of the time segments of the time window of said previous action to obtain a time window of the end times of said previous action;
shifting the time segments of the time window of the end times of said action backward by the duration of said action to obtain a time window of the start times of said action;
obtaining a time window of the start times of said action of which the segments are the cross section of the segments of the time window of the end times of the previous action and the segments of the time window of the start times of said action.

5. The method according to claim 11, wherein the inductive step of obtaining a time window of end times of an action by processing a time window of said action, a time window of a following action and a slack time comprises the following steps:
subtracting the duration from the end times of the time segments of the time window of said following action to obtain a time window of start times of said following action;
adding the slack time to the end times of the time segments of the time window of said action to obtain a time window of said action plus slack;
adding the duration to the start times of the time segments of the time window of said action plus slack to obtain a time window of end times of said action;
obtaining a time window of end times of said action of which the time segments are the cross section of the time segments of a time window of start times of said following action and the time segments of a time window of end times of said action.

6. The method of claim 12, wherein the inductive step of obtaining a time window of an action by processing the time windows of actions that are either a parent action or related actions comprises the following steps
replacing the time segments of time windows of related actions by time segments that are the cross sections of the original time segments of said time windows of said related actions;
adjusting the begin and end times of time segments of children actions so that are comprised in the time segments of the time window of the parent action.

7. A method according to claim 1, wherein at least one of said resources is a patient, a physician, a nurse, a medical diagnostic apparatus, a medical treatment apparatus, an examination room or a treatment room.

8. A data processing system comprising means for carrying out the steps of the method according to claim 1.

9. A computer program comprising computer program means adapted to perform the method according to claim 1, when said computer program is executed on a computer.

10. A computer readable medium comprising program code adapted to carry out the method according to claim 1, when executed on a computer.

11. The method according to claim 1, wherein the inductive logic step comprises obtaining a time window of end times of an action by processing a time window of said action, a time window of a following action and a slack time.

12. The method according to claim 1 wherein the inductive logic step comprises obtaining a time window of an action by processing the time windows of actions that are either a parent action or related actions.
